(19) United States
(12) Patent Application Publication
(10) Pub. No.: US 2024/0083974 A1
(43) Pub. Date: Mar. 14, 2024

(54) Artificial Transmembrane Proteins for Detecting Intracellular or Intravesicular Biomolecular Interactions

(71) Applicants: ETH Zürich, Zürich (CH); Hoffmann-La Roche Inc., Little Falls, NJ (US)

(72) Inventors: Janos Voeroes, Zürich (CH); Yves Lukas Blickenstorfer, Zürich (CH); Christof Fattinger, Basel (CH); Andreas Frutiger, Zürich (CH); Andreas Michael Reichmuth, Zürich (CH); Ines Lüchtefeld, Zürich (CH); Ilaria Incaviglia, Zürich (CH)

(21) Appl. No.: 17/767,810

(22) PCT Filed: Oct. 13, 2020
(86) PCT No.: PCT/EP2020/078797

§ 371 Date: Apr. 8, 2022

(30) Foreign Application Priority Data
Oct. 15, 2019 (EP) 19203205.0

(51) Int. Cl. C07K 14/72; C12N 15/63; G01N 33/533

(52) U.S. Cl. CPC C07K 14/723; C12N 15/63; G01N 33/533; C07K 2319/02; C07K 2319/03; C07K 2319/20; C07K 2319/33; C07K 2319/60

(57) ABSTRACT

Disclosed herein is an artificial transmembrane protein for use in a biomolecular detection device for detecting intracellular or intravesicular biomolecular interactions, the artificial transmembrane protein having an extracellular or extravesicular binder structure, a hydrophobic transmembrane domain, and an intracellular or intravesicular domain with an intracellular or intravesicular receptor structure, wherein the receptor structure is configured to interact with an intracellular or intravesicular component of the biomolecular interaction to be detected and wherein the extracellular or extravesicular binder structure is configured to bind to membrane recognition elements arranged along a plurality of predetermined lines of the biomolecular detection device.

### CROSS-REFERENCE TO RELATED APPLICATIONS

This application is the United States national phase of International Application No. PCT/EP2020/078797 filed Oct. 13, 2020, and claims priority to European Patent Application No. 19203205.0 filed Oct. 15, 2019, the disclosures of which are hereby incorporated by reference in their entirety.

The Sequence Listing associated with this application is filed in electronic format via EFS-Web and is hereby incorporated by reference into the specification in its entirety. The name of the text file containing the Sequence Listing is 2201508 ST25.txt. The size of the text file is 16,420 bytes, and the text file was created on Mar. 9, 2022.

### BACKGROUND

### Field of Disclosure

The present invention lies in the field of artificial transmembrane proteins, in particular for detecting intracellular or intravesicular biomolecular interactions. The invention is further directed to a biomolecular detection device for analyzing a cell, vesicle or a cellular or vesicular component comprising an artificial transmembrane protein as well as to a method for detecting intracellular or intravesicular biomolecular interactions in a cell, cellular component or a vesicle or vesicular component.

### Background

Detection devices are used, for example, as biosensors in a large variety of applications. One particular application is the detection or monitoring of binding affinities or processes. For example, with the aid of such biosensors various assays detecting the binding of target samples to binding sites can be performed. Typically, large numbers of such assays are performed on a biosensor at spots which are arranged in a two-dimensional microarray on the surface of the biosensor. The use of microarrays provides a tool for the simultaneous detection of the binding affinities or processes of different target samples in high-throughput screenings. For detecting the affinities of target samples to bind to specific binding sites, the affinity of target molecules to bind to specific capture molecules, a large number of capture molecules are immobilized on the outer surface of the biosensor at individual spots (e.g. by ink-jet spotting or photolithography). Each spot forms an individual measurement zone for a predetermined type of capture molecule. The binding of a target molecule to a specific type of capture molecule is detected and is used to provide information on the binding affinity of the target molecule with respect to the specific capture molecule.

A known technique for detecting binding affinities of target samples utilizes fluorescent labels. The fluorescent labels are capable of emitting fluorescent light upon excitation. The emitted fluorescent light has a characteristic emission spectrum which identifies the present fluorescent label at a particular spot. The identified fluorescent label indicates that the labelled target molecule has bound to the particular type of binding sites present at this spot.

A sensor for detecting labelled target samples is described in the article “Zeptosens' protein microarrays: A novel high performance microarray platform for low abundance protein analysis”, Proteomics 2002, 2, S. 383-393, Wiley-VCH Verlag GmbH, 69451 Weinheim, Germany. The sensor described therein comprises a planar waveguide arranged on a substrate. The planar waveguide has an outer surface capable of attaching a plurality of binding sites thereon. Moreover, the planar waveguide has a plurality of incoupling lines for coupling a beam of coherent light into the planar waveguide in a manner such that a beam of coherent light propagates along the planar waveguide. The coherent light propagates through the planar waveguide under total internal reflection with an evanescent field of the coherent light propagating along the outer surface of the planar waveguide. The depth of penetration of the evanescent field into the medium of lower refractive index at the outer surface of the planar waveguide is in the order of magnitude of a fraction of the wavelength of the coherent light propagating through the planar waveguide. The evanescent field excites the fluorescent labels of the labelled target samples bound to the binding sites arranged on the surface of the planar waveguide. Due to the very small depth of penetration of the evanescent field into the optically thinner medium at the outer surface of the planar waveguide, only the labelled samples bound to the binding sites immobilized on the outer surface of the planar waveguide are excited. The fluorescent light emitted by these labels is then detected with the aid of a CCD camera.

While it is principally possible to detect the binding affinities using fluorescent labels, this technique is disadvantageous in that the detected signal is produced by the fluorescent labels rather than by the binding partners themselves. In addition, labelling the target samples requires additional preparation steps. Moreover, labelled target samples are comparatively expensive. Another disadvantage is the falsification of the results caused by steric hindrance of the fluorescent labels at the target sample which might interfere with the binding of the target samples to the capture molecules. Further disadvantages are the falsification of the results due to photobleaching of the labels or quenching effects. In addition, fluorescent labelling may significantly influence the chemical, biological, pharmacological and physical properties of the compound of interest. Thus, measurements relying solely on fluorescent labelling may be falsified by the presence of such a label. Furthermore, fluorescence spectroscopy requires that any compound or cellular component of interest is labelled. Thus, it is only possible to observe interactions with the particular compound labelled, but not any further interactions.

### SUMMARY

A crucial requirement for analyzing biological samples is the discrimination between specific binding of a compound or structural moiety of interest to a binding site from non-specific binding. Known strategies for addressing this issue such as surface plasmon resonance (SPR) or Mach Zehnder interferometry rely strongly on reference measurements and are only suitable for measurements under static conditions. Thus, such techniques are typically not suitable for measurements of complex environments, such as detection of biomolecular interactions within a living cell. SPR measures the refractive index change upon receptor-ligand binding in the vicinity of the sensor surface. This technique however has the disadvantage that it is susceptible to any refractive index change in vicinity of the sensor surface. Therefore, non-specific binding still represents a significant problem. In particular, refractometric sensors are unable to provide any distinction between molecules that actually bind to the target of interest and the sole presence of other compounds and additional effects effecting the refractive index.

As G-protein coupled receptors (GPCRs) have evolved as particular prominent targets for drug candidates, due to their involvement in the development and progression of many diseases such as pain, asthma, inflammation, obesity and cancer, detailed analysis of these receptors in living cells are highly desirable. Whole cell assays to determine GPCR activity traditionally rely on the detection of distinct intracellular second messengers (cAMP, Ca²⁺), relocalization of fluorescently tagged proteins (arrestin recruitment to the receptor or receptor internalization) or the expression of a reporter gene under control of a GPCR-activated signaling cascade. However, as pointed out above, fluorescent labelling requires considerable biomolecular modifications, such as the overexpression of proteins or introduction of fluorescent labels, which are not always possible or desirable as these can alter cellular physiology or drug pharmacology. Furthermore, GPCRs typically modulate more than one effector which can lead to cross sensitivity in these assays.

Label-free cellular assays, like the resonant waveguide grating biosensor, do not require any molecular labels (see Paulsen et al. Photonics Nanostruct. Fundam. Appl. 2017, 26, 69). Generally, such refractometric sensors monitor the refractive index above the sensor chip by means of a propagating evanescent wave which by its penetration depth defines the sensing volume. Redistribution of cellular content within this sensing volume results in an overall change in the refractive index, giving rise to the much-appreciated holistic picture of dynamic mass redistribution (DMR). In return, DMR is inherently cross sensitive, meaning different GPCRs mediated signaling pathways cannot be deconvolved spatiotemporally by the sensor.

As an alternative to optical label-free whole cell analysis, such as SPR, dynamic mass redistribution based on resonant waveguide grating (RWG), symmetry waveguide sensors and quantitative phase imaging have been used to investigate morphological changes and phenotypic cellular responses for drug discovery. Noteworthy however, these methods only enable to provide information about the cytosolic mass, solute concentration and volume changes, while intracellular or intravesicular processes cannot be monitored.

It is therefore an overall object of the present invention to improve the state of the art regarding the detection of intracellular or intravesicular interactions, thereby preferably avoiding the disadvantages of the prior art fully or partly.

In favorable embodiments, an artificial transmembrane protein is provided, which can be specifically engineered such that it is configured for interacting with a specific intracellular or intravesicular component and concomitantly enables to monitor a direct or indirect interaction with said intravesicular or intracellular component.

In further favorable embodiments, an artificial transmembrane protein is provided that enables single cell measurements.

The overall objective is in a general way achieved by the subject-matter of the independent claims. Further advantageous and exemplary embodiments follow from the description and the figures.

According to a first aspect of the invention, the overall objective is achieved by an artificial transmembrane protein for use in a biomolecular detection device for detecting intracellular or intravesicular biomolecular interactions. The artificial transmembrane protein comprises an extracellular or extravesicular binder structure, a hydrophobic transmembrane domain and an intracellular or intravesicular domain comprising an intracellular or intravesicular receptor structure. The receptor structure is configured to interact with an intracellular or intravesicular component of the biomolecular interaction to be detected and wherein the extracellular or extravesicular binder structure is configured to bind to membrane recognition elements arranged along a plurality of predetermined lines of the biomolecular detection device. It is understood that interaction of the receptor structure with the intracellular or intravesicular component of the biomolecular interaction to be detected refers to an interaction in the biomolecular sense. Thus, such an interaction may for example be a binding of a compound to the receptor. The intracellular or intravesicular receptor structure can be specifically designed, for example by genetic engineering, to interact with any given intracellular or intravesicular component of interest. Therefore, the artificial transmembrane proteins are biomimetic membrane receptors, which are designed by available biotechnological procedures for a specific purpose.

Preferably, the biomolecular detection device which may be used in combination with an artificial transmembrane protein according to any of the embodiments described herein for detecting the biomolecular interaction of interest comprises an evanescent illuminator with an optical coupling unit configured for generating an evanescent field from coherent light with a predefined wavelength on a first surface of the evanescent illuminator. The first surface of the evanescent illuminator comprises a template nanopattern, which contains a coherent arrangement of a plurality of predetermined lines along which membrane recognition elements for a binder structure of an artificial transmembrane protein, preferably a laterally diffusible artificial transmembrane protein of the cell, vesicle or the cellular or vesicular component are arranged. A laterally diffusible transmembrane protein is a transmembrane protein that may diffuse within the membrane of a cell or vesicle. The membrane recognition elements are configured to bind the binder structure of the laterally diffusible artificial transmembrane protein for forming a transmembrane nanopattern within the cell, vesicle or the cellular or vesicular component based on the template nanopattern of the evanescent illuminator by locally locking the artificial transmembrane proteins, such that light of the evanescent field is scattered by the cell, vesicle or the cellular or vesicular component bound to the membrane recognition elements. As the skilled person understands, the evanescent illuminator is an element which is able to generate an evanescent field from coherent light of a light source. The predetermined lines on the evanescent illuminator are arranged such that light scattered by the cell, vesicle or cellular or vesicular components bound to the membrane recognition elements constructively interferes at a predefined detection site with a difference in optical path length that is an integer multiple of the predefined wavelength of the coherent light. As the skilled person understands, the optical path length refers to the product of the geometric length of the path followed by light through a given system, and the index of refraction of the medium through which it propagates. As the extracellular or extravesicular binder structure of the artificial transmembrane protein is configured to bind to membrane recognition elements arranged along a plurality of predetermined lines of the biomolecular detection device, the template nanopattern of the biomolecular detection device is transferred into a living cell, by selectively binding transmembrane proteins which are laterally diffusible within the cell, thus generating a transmembrane nanopattern in the cell itself. Typically, membrane recognition elements of multiple different predetermined lines bind to the same cell, i.e. more than one membrane recognition element binds to or is configured to bind to a single cell, vesicle or cellular or vesicular component.

Due to the fact that the extracellular or extravesicular binder structure is configured to bind to membrane recognition elements arranged along the plurality of predetermined lines of the biomolecular detection device and that the receptor structure can be specifically designed for example by genetic engineering to interact with any given intracellular or intravesicular component of interest, the artificial transmembrane proteins provide a detection windows that are specific for the biomolecular detection of interest. The template nanopattern of the biomolecular detection device allows for transferring or reproducing the template nanopattern of the biomolecular detection device into a living cell membrane, by selectively binding artificial transmembrane proteins which are laterally diffusible within the cell. As a result, any biomolecular interaction, even intercellular processes, that involve an interaction of the artificial transmembrane protein can be selectively detected. Upon binding between the membrane recognition elements and the binder structure of the artificial transmembrane protein, evanescent light is scattered and constructively interferes at a predefined detection site. The constructive interference of the scattered light relates to all bound transmembrane proteins and yields quadratic scaling of the measured intensity with respect to the number of transmembrane proteins. Importantly, random scattering of background molecules which are not bound to the molecular recognition elements interfere with equal probability both constructively and destructively. As a result, cross sensitivity is significantly reduced. Surprisingly, even though living cells and also vesicles comprise highly uneven surfaces on the nanometer scale, hardly any cross-sensitivity is observed due to any cell scattering, particularly membrane scattering. While measurements of living cells may be readily disturbed by signals overruling the light scattered at the bound membrane recognition elements, no significant loss in sensitivity and only minor distortion of the focused diffracted signal has been observed.

Typically, the coherent light has a predetermined wavelength and is preferably monochromatic, particularly at a single wavelength. Usually, visible or near infrared light may be used. A portion of the evanescent field is scattered coherently by scattering centers composed of biomolecules from cells, vesicles or cellular or vesicular components bound to the membrane recognition elements which are arranged on the different predetermined lines. The scattered electric field at any location can be determined by adding the contributions from each of the individual scattering centers and then computing the intensity by squaring the resulting phasor. A maximum of the scattered intensity is located at the predetermined detection location because the predetermined lines are arranged such that at the predetermined detection location, the optical path length of the light scattered by the different scattering centers differs by an integer multiple of the wavelength of the light. The requirement of constructive interference is met by any scattered light which adds to the detectable signal in the detection location. The intensity pattern at the predetermined detection location preferentially forms but is not limited to a diffraction limited Airy disk. In essence, any shape accessible by Fourier optics is possible. For any shape, the signal is best recoverable with a matched filter.

The scattering of an isolated membrane recognition element—transmembrane complex with refractive index n_{R} embedded in a medium with refractive index no in plane-polarized light is:

$$\begin{matrix}\frac{{I}_{scan} ( r )}{{I}_{R}} = \frac{9 {π}^{2} {{n}_{0}}^{4}}{{r}^{2} {λ}^{4}} {\vert \frac{{{n}_{R}}^{2} - {{n}_{0}}^{2}}{{{n}_{R}}^{2} + 2 {{n}_{0}}^{2}} \vert}^{2} {{V}_{R}}^{2} \\ = \frac{9 {π}^{2} {{n}_{0}}^{4}}{{r}^{2} {λ}^{4}} {( \frac{dn}{dc} )}^{2} \frac{{{M}_{R}}^{2}}{{{N}_{A}}^{2}} \tilde{n} ( {ρ}_{R} ) ,\end{matrix}$$

wherein
r is the distance from the compound of interest to the detection site
□ is the vacuum wavelength
I_{R} is the intensity of the plane-polarized field incident on the artificial transmembrane complex
V_{R} the volume of the artificial transmembrane complex;
and wherein

$${V}_{R} = \frac{dn}{dc} \frac{{M}_{R}}{{N}_{A} ( {n}_{R} - {n}_{0} )} ; \tilde{n} ( {ρ}_{R} ) = \frac{{( {n}_{R} + {n}_{0} )}^{2}}{{( {{n}_{R}}^{2} + 2 {{n}_{0}}^{2} )}^{2}} .$$

M_{R} is the molar mass of the artificial transmembrane complex
N_{A} is the Avogadro constant.

$$\frac{dn}{dc}$$

is the refractive index increment of the compound of interest.

The total mass density Γₜₒₜ and through a straightforward manipulation also the number of receptors can be computed from the measured intensity at the center of the Airy disk at the predetermined detection location by:

$${Γ}_{tot} = \frac{{λ}^{2}}{{π}^{2} NA \frac{dn}{dc} {η}_{[ A ]} D} \sqrt{\frac{{I}_{{Airy}_{max}}}{{I}_{0}}}$$

wherein
N_{A} is the numerical aperture of the circular template nanopattern
η_{[A]} is the analyte efficiency of the fabricated template nanopattern
D is the diameter of the circular template nanopattern
I₀ is the average intensity incident on the ensemble of receptors
I_{Airy}_{ₘₐₓ} intensity at the center of the Airy disk, namely the predetermined detection location.

Thus, depending on experimental design, the intensity change of the scattered light provides access to the molecular mass of an interaction partner, the total bound mass or the number of receptors involved during a biomolecular interaction in which the artificial transmembrane protein is involved. For example, if a certain messenger compound binds to the artificial transmembrane protein the corresponding mass increase can be calculated. For example, the intracellular or intravesicular receptor structure may be configured to interact with a first component A of a biomolecular interaction, which has a known molecular mass. Upon binding of component A, a change in the diffracted intensity is observed. This intensity change can be used to compute the number of receptors that interact with component A. When a second component B binds to component A or also to the receptor structure, a further change of intensity is observed. When component A and/or B is then unbound from the receptor structure, the mass of the corresponding complex decreases again and the intensity of the signal changes. Under the assumption that B binds to the same amount of binding sites as A did, the molecular mass of the complex as well as the one of B can be calculated at any given time of the measurement, thereby allowing real-time monitoring of the biomolecular interaction over time. Due to the mass difference, it is possible to obtain information on any compound that is directly or indirectly bound or unbound to or from the artificial transmembrane protein.

In some embodiments, the artificial transmembrane protein further comprises a linker domain configured to facilitate the interaction between the intracellular or intravesicular receptor structure and the intracellular or intravesicular component of the biomolecular interaction to be detected, wherein the linker domain is arranged between the intracellular or intravesicular receptor structure and the hydrophobic transmembrane domain. Preferably, the linker domain is free of bulky hydrophobic residues, such as tryptophan, which may interfere with protein folding. Thus, the linker domain may consist of the amino acids glycine, serine, alanine, glutamine, proline and phenylalanine or only of glycine. Small amino acids such as glycine may provide higher flexibility of the linker domain. Polar resides, such as glutamine increase solubility of the linker in water. The linker domain may in general have a length of 4 to 25 residues, preferably 4 to 20 residues.

In further embodiments, the extracellular or extravesicular binder structure is configured to establish a covalent bond to the membrane recognition elements arranged along a plurality of predetermined lines of the biomolecular detection device. For example, the extracellular or extravesicular binder structure may comprise a chemical moiety that enables the generation of a covalent bond, such as an electrophile, a nucleophile, a dienophile, a diene, a 1,3-dipole or a dipolarophile.

In certain embodiments, the extracellular or extravesicular binder structure comprises a nucleophile, preferably a thiol or a thiolate.

In further embodiments, the extracellular or extravesicular binder structure comprises or consists of a SNAP tag or a CLIP tag. The SNAP tag is a 182 residues polypeptide and accepts O⁶-benzylguanine derivatives (19.4 kDa, Crivat et al. Trends in Biotechnology. 30 (1): 8-16. doi: 10.1016/j.tibtech.2011.08.002; Juillerat et al. Chemistry and Biology. 10 (4): 313-317, doi:10.1016/51074-5521(03)00068-1; Molliwtz et al. Biochemistry. 51 (5): 986-994. doi:10.1021/bi2016537). The CLIP tag is related to the SNAP tag and has been engineered to accept O²-benzylcytosine derivatives as substrates, instead of O⁶-benzylguanine (Gautier et al. Chemistry and Biology. 15 (2): 128-136. doi: 10.1016/j.chembio1.2008.01.007).

In some embodiments, the artificial membrane protein is a single-pass transmembrane protein.

In further embodiments, the artificial transmembrane protein is
(a) of type I, wherein the intracellular or intravesicular domain is arranged adjacent to the C terminus and the extracellular or extravesicular domain is arranged adjacent to the N terminus; or
(b) of type II wherein the intracellular or intravesicular domain is arranged adjacent to the N terminus and the extracellular or extravesicular domain is arranged adjacent to the C terminus.

In an artificial transmembrane protein of type I, the N-terminal is configured to face the cell exterior, while the C-terminal is configured to remain in the cytosol. In contrast, in an artificial transmembrane protein of type II, the N-terminal is configured to remain in the cytosol, while the C-terminal is configured to face the cell exterior.

In more specific embodiments of type I, the order of domains from the N to the C terminus may be: the extracellular or extravesicullar binder structure, preferably with a SNAP or CLIP tag, followed by the hydrophobic transmembrane domain, which is optionally followed by the linker domain, followed by the intracellular or intravesicular domain with the intracellular or intravesicular receptor structure. Preferably, positively charged amino acids are arranged before the linker domain. Optionally, the extracellular or extravesicullar binder structure comprises a cleavable signal peptide which may preferably be arranged in front of the SNAP or CLIP tag, i.e. being closest to the N terminus.

In more specific embodiments of type II, the order of domains from the N to the C terminus may be: the intracellular or intravesicular domain with the intracellular or intravesicular receptor structure, which is optionally followed by the linker domain, followed by the hydrophobic transmembrane domain, followed by the extracellular or extravesicullar binder structure, preferably with a SNAP or CLIP tag. Optionally, the intracellular or intravesicular domain may comprise a cleavable signal peptide which may preferably be arranged in front of the receptor structure.

In some embodiments, the artificial transmembrane protein is of type I or II and the intracellular or intravesicular domain comprises a higher amount of positively charged amino acid residues than the extracellular or extravesicular domain. In particular, the positively charged amino acids may be selected from lysine, arginine or histidine, preferably lysine. In particular, these amino acids may occur in the intracellular or intravesicular 3 to 4 times more often than in the rest of the artificial transmembrane protein. The higher amount of positively charged amino acids enables a more efficient orientation of the artificial transmembrane helices within the membrane.

In further embodiments, the intracellular or intravesicular receptor structure is configured to interact of a β subunit of protein kinase in the cAMP pathway or with the receptor tyrosine kinase (RTK). If the intracellular or intravesicular receptor structure is configured to interact with RTK, the receptor structure may comprise Grb2 protein.

In some embodiments, the intracellular or intravesicular receptor structure may comprise a fluorescent protein, such as eYFP.

In some embodiments, the intracellular or intravesicular receptor structure is a designed receptor, artificial binder or other functional molecule, such as antibody, antibody fragment, nanobody, affimer, or the like.

In further embodiments, the artificial transmembrane protein comprises a cleavable signal peptide adjacent the N terminus of the artificial transmembrane protein for interaction with a protein transport system and for controlling translocation of the artificial transmembrane protein.

Typically, the cleavable signal peptide consists of 18-26 amino acids, which may form a positively charged N-terminal n-region, a central hydrophobic h-region and a polar C-terminal c-region. Preferably, the cutting site is contained in the c-region and is configured to be recognized by signal peptidase.

In some embodiments, the extracellular or extravesicullar binder structure comprises an affinity tag configured for interacting, preferably selectively interacting with the membrane recognition elements of the biomolecular detection device. For example, the affinity tag may be a HA (human influenza hemagglutinin), FLAG or 6His affinity tag. The use of affinity tags is beneficial, as the protein conformation may be altered by molecular interactions, which may limit or prevent antibodies acting as membrane recognition elements from recognizing the extracellular or extravesicular binder structure of the artificial transmembrane protein. This can be avoided by affinity tags in the extracellular or extravesicullar binder structure.

In some embodiments the artificial transmembrane protein comprises an intracellular or intravesicular fluorescent protein or a protein configured for interacting with other intracellular elements, such as a biomolecular tag. For example, for intracellular sortase mediated immobilization of proteins. Preferably, the protein is configured for specifically interacting with other intracellular elements.

In some embodiments, the transmembrane protein is label-free, in particular fluorescent label-free. Preferably, the transmembrane protein does not contain an artificial label moiety which can be excited upon irradiation. A fluorescent label-free transmembrane protein as used herein is a transmembrane protein which does not contain a small fluorescent molecule, i.e. a molecule having a molecular weight of below 900 Da.

According to a further aspect, the invention is directed to a cell, vesicle or cellular or vesicular component comprising an artificial transmembrane protein according to any the embodiments as described herein or a nucleic acid sequence encoding an artificial transmembrane protein according to any of the embodiments as described herein. The nucleic acid sequence can be introduced, preferably in vitro, into a cell and expressed therein, for use in a biomolecular detection device. According to a further aspect, the invention is directed to a recombinant nucleic acid molecule comprising at least one nucleic acid sequence encoding an artificial transmembrane protein according to any of the embodiments as described herein.

According to a further aspect, the invention is directed to a vector, preferably a plasmid vector, comprising the recombinant nucleic acid molecule as described in any embodiments herein.

According to a further aspect, the invention is directed to a use of a vector according to any of the embodiments described herein for expressing an artificial transmembrane protein in vitro, comprising:
providing a cell and introducing a vector according to any of the embodiments described herein in the cell, and expressing the artificial transmembrane protein.

In some embodiments, the use further comprises the step of cleaving a signal peptide from the artificial transmembrane protein.

According to a further aspect, the invention is directed to a biomolecular detection device for analyzing a cell, vesicle or a cellular or vesicular component comprising an artificial transmembrane protein according to any of the embodiments as described herein□, the biomolecular detection device comprising an evanescent illuminator with an optical coupling unit configured for generating an evanescent field from coherent light with a predefined wavelength on a first surface of the evanescent illuminator, the first surface of the evanescent illuminator comprising a template nanopattern, containing a coherent arrangement of a plurality of predetermined lines along which membrane recognition elements for a binder structure of the artificial transmembrane protein, of the cell, vesicle or the cellular or vesicular component are arranged, wherein the membrane recognition elements are configured to bind the binder structure of the artificial transmembrane protein for forming a transmembrane nanopattern within the cell, vesicle or the cellular or vesicular component based on the template nanopattern of the evanescent illuminator, such that light of the evanescent field is scattered by the cell, vesicle or the cellular or vesicular component bound to the membrane recognition elements, and wherein the predetermined lines are arranged such that light scattered by the cell, vesicle or cellular or vesicular components bound to the membrane recognition elements constructively interferes at a predefined detection site with a difference in optical path length that is an integer multiple of the predefined wavelength of the coherent light.

The template nanopattern allows for transferring the template nanopattern of the biomolecular detection device into a living cell, by selectively binding transmembrane proteins which are laterally diffusible within the cell, thus generating a transmembrane nanopattern in the cell itself. Typically, membrane recognition elements of multiple different predetermined lines bind to the same cell, i.e. more than one membrane recognition element binds to or is configured to bind to a single cell, vesicle or cellular or vesicular component. As a result, any biomolecular interaction, even intercellular or intracellular processes, that involve an interaction of the transmembrane protein can be selectively detected. Upon binding between the membrane recognition elements and the binder structure of the transmembrane protein, evanescent light is scattered and constructively interferes at a predefined detection site. The constructive interference of the scattered light relates to all bound transmembrane proteins and yields quadratic scaling of the measured intensity with respect to the number of transmembrane proteins. Importantly, random scattering of background molecules which are not bound to the molecular recognition elements interfere with equal probability both constructively and destructively. As a result, cross sensitivity is effectively suppressed. Surprisingly, even though living cells and also vesicles comprise highly uneven surfaces on the nanometer scale, hardly any cross sensitivity is observed due to any cell scattering, particularly membrane scattering. While measurements of living cells may be readily disturbed by signals overruling the light scattered at the bound membrane recognition elements, no significant loss in sensitivity has been observed. This is surprising, as the cells, vesicles or cellular or vesicular components are significantly larger than the distances between the predetermined lines of the nanopattern. Thus, in general in the embodiments disclosed herein, the membrane recognition elements of multiple different predetermined lines bind to the same cell. Typically, the distance between two directly adjacent predetermined lines is between 2 to 100 times smaller, preferably 30 to 100 times smaller, as the single cell.

In some embodiments, the evanescent illuminator comprises or is a carrier with a planar waveguide arranged on a surface of the carrier and an optical coupler as the optical coupling unit for coupling coherent light of a predefined wavelength into the waveguide such that the coherent light propagates through the planar waveguide with an evanescent field of the coherent light propagating along a first surface of the planar waveguide and wherein the first surface of the planar waveguide comprising the template nanopattern.

In some embodiments, the evanescent illuminator is a total internal reflection system configured for providing a beam of coherent light at the predetermined wavelength and at a predetermined angle onto the first surface of the evanescent illuminator by means of the optical coupling unit, particularly by a prism or any other suitable optical element.

In some embodiments, the plurality of predetermined lines comprises curved lines with a curvature configured such that light of the evanescent field scattered by the cell, vesicle or the vesicular or cellular component, or biomolecules thereof, bound to the membrane recognition elements interferes at the predefined detection site.

Preferably, the overall shape of the template nanopattern may be round, particularly circular.

In preferred embodiments, the curved lines are arranged with a decreasing distance between adjacent lines in the propagation direction of the light for focusing the light scattered by the cell, vesicle or cellular or vesicular component into the predetermined detection site. Alternatively, the plurality of predetermined lines may comprise straight lines being arranged with a predefined angle to the propagation of the light coupled to the evanescent illuminator. An additional coupler may be employed for focusing the diffracted light into the predefined detection site.

In further embodiments, the plurality of predetermined lines are arranged on the outer surface of the evanescent illuminator in a manner such that their locations in xⱼ,yⱼ coordinates are geometrically defined by the equation:

$${x}_{j} = \frac{λ N ( {A}_{0} + j ) - \sqrt{{n}_{S}^{2} ( {N}^{2} - {n}_{S}^{2} ) ( {y}_{j}^{2} + {f}^{2} ) + {( {n}_{S} λ )}^{2} {( {A}_{0} + j )}^{2}}}{{N}^{2} - {n}_{S}^{2}}$$

wherein
λ is the vacuum wavelength of the propagating light;
N is the effective refractive index of the guided mode in the planar waveguide; N depends on the thickness of the planar waveguide, the refractive index of the carrier, the refractive index of a medium on the first surface of the planar waveguide and the polarization of the guided mode; In the case of total internal reflection systems, N can be expressed via the angle of total internal reflection θ_{TIR}:N=nₛ sin(θ_{TIR}).
nₛ is the refractive index of the carrier
f is the focal distance of the predetermined detection location;
A₀ is an integer which is chosen to be close to the product refractive index nₛ and the focal distance of the predetermined detection location f of the carrier divided by the wavelength λ; and
j is a running integer indicating the index of the respective line.

The chosen integer A₀ assigns negative x-values at the center of the lines with negative j values and positive x-values at the center of lines with positive j values. Or to say it in other words, the integer A₀ defines the origin of the x,y coordinates frame that is used for the location of the lines at the outer surface of the evanescent illuminator; the chosen A₀ value puts the detection location at x=0, y=0, z=−f.

In some embodiments, at least one cell, vesicle or cellular or vesicular component is bound via the binder structure of the artificial transmembrane protein to the membrane recognition elements.

According to another aspect, the invention is directed to a kit of parts, comprising an artificial transmembrane protein according to any of the embodiments as described herein, or a cell according to any of the embodiments as described herein, or a recombinant nucleic acid molecule according to any of the embodiments as described herein, or a vector according to any of the embodiments as described herein, and a biomolecular detection device according to any of the embodiments as described herein.

In some embodiments, the kit of parts further comprises a protein of interest configured for intracellular or intravesicular biomolecular interaction, wherein the protein of interest comprises a high-mass moiety, in particular a gold nanoparticle. The use of such high-mass moieties is beneficial, as the higher molecular mass has a beneficial effect on the intensity of the obtained signal, thus even enabling single cell measurements. As the skilled person understands, a high-mass moiety typically has a significantly larger molecular weight than the artificial transmembrane protein. For example, the high-mass moiety may also be a protein or an overexpressed protein aggregate. Preferably, the mass of the high-mass moiety may be at least 150 kD.

According to another aspect, the invention is directed to a label-free method for detecting intracellular or intravesicular biomolecular interactions in a cell, cellular component or a vesicle or vesicular component using a biomolecular detection device according to any of the embodiments as described herein, the method comprising the steps:
Optionally providing a cell, cellular component or vesicle or vesicular component or vesicular component comprising an artificial transmembrane protein according to any of the embodiments as described herein;
Applying the cell, cellular component or vesicle or vesicular component to the membrane recognition elements of the biomolecular detection device or providing a biomolecular detections device with at least one cell, vesicle or cellular or vesicular component is bound via the binder structure of the artificial transmembrane protein to the membrane recognition elements;
Generating a beam of coherent light at a predefined beam generation location relative to the plurality of predetermined lines, the beam of coherent light having a predefined wavelength and being incident on the membrane recognition elements with the bound transmembrane protein in a manner that diffracted portions of the incident beam of coherent light constructively interfere at the predefined detection site relative to the plurality of predetermined lines with a difference in optical path length that is an integer multiple of the predefined wavelength of the coherent light to provide a signal representative of the membrane recognition elements with the artificial transmembrane protein of a cell, vesicle or cellular or vesicular component bound thereto at the predefined detection site;
Measuring the signal representative for the membrane recognition elements with the artificial transmembrane protein of a cell, vesicle or cellular or vesicular component bound thereto.

A label-free method for detecting intracellular or intravesicular biomolecular interactions in a cell, cellular component or a vesicle or vesicular component is a method which does not rely on an interaction between light of a light source, particularly coherent light, and a label. Such a label is for example an artificial label moiety which can be excited upon irradiation, such as a fluorescent label. Commonly used fluorescent labels are small fluorescent molecules, i.e. a fluorescent molecule having a molecular weight of below 900 Da.

In some embodiments, the method further comprises the step of providing a substance that interacts with the receptor structure of the artificial transmembrane protein or with intracellular or intravesicular component of the biomolecular interaction to be detected, such as a drug, drug candidate, small molecule or an antibody.

In certain embodiments, the cell or cellular component comprising the artificial transmembrane protein is provided by in vitro transfecting a cell with a vector according to any of the embodiments as described herein, expressing the artificial transmembrane protein in the cell and optionally removing parts of the membrane of the cell for providing the cellular component.

In some aspects, the invention is described by the following clauses:
Clause 1: An artificial transmembrane protein for use in a biomolecular detection device for detecting intracellular or intravesicular biomolecular interactions, the artificial transmembrane protein comprising an extracellular or extravesicular binder structure, a hydrophobic transmembrane domain and an intracellular or intravesicular domain with an intracellular or intravesicular receptor structure, wherein the receptor structure is configured to interact with an intracellular or intravesicular component of the biomolecular interaction to be detected and wherein the extracellular or extravesicular binder structure is configured to bind to membrane recognition elements arranged along a plurality of predetermined lines of the biomolecular detection device.
Clause 2: The artificial transmembrane protein according to clause 1, further comprising a linker domain configured to facilitate the interaction between the intracellular or intravesicular receptor structure and the intracellular or intravesicular component of the biomolecular interaction to be detected, wherein the linker domain is arranged between the intracellular or intravesicular receptor structure and the hydrophobic transmembrane domain.
Clause 3: The artificial transmembrane protein according to any of clauses 1 or 2, wherein the extracellular or extravesicular binder structure is configured to establish a covalent bond to the membrane recognition elements arranged along a plurality of predetermined lines of the biomolecular detection device.
Clause 4: The artificial transmembrane protein according to any of the previous clauses, wherein the extracellular or extravesicular binder structure comprises a nucleophile, preferably a thiol or a thiolate.
Clause 5: The artificial transmembrane protein according to any of the previous clauses, wherein the extracellular or extravesicular binder structure is a SNAP tag or a CLIP tag.
Clause 6: The artificial transmembrane protein according to any of the previous clauses, wherein the artificial transmembrane protein is
(a) of type I, wherein the intracellular or intravesicular domain is arranged adjacent to the C terminus and the extracellular or extravesicular domain is arranged adjacent to the N terminus; or
(b) of type II wherein the intracellular or intravesicular domain is arranged adjacent to the N terminus and the extracellular or extravesicular domain is arranged adjacent to the C terminus.
Clause 7: The artificial transmembrane protein according to clause 7, wherein the artificial transmembrane protein is of type II and wherein the intracellular or intravesicular domain comprises a higher amount of positively charged amino acid residues than the extracellular or extravesicular domain.
Clause 8: The artificial transmembrane protein according to any of the preceding clauses, wherein the intracellular or intravesicular receptor structure is a designed receptor or other functional molecule, such as antibody, antibody fragment, nanobody or affimer.
Clause 9: The artificial transmembrane protein according to any of the previous clauses, further comprising a cleavable signal peptide adjacent the N terminus of the artificial transmembrane protein for interaction with a protein transport system and for controlling translocation of the artificial transmembrane protein.
Clause 10: The artificial transmembrane protein according to any of the previous clauses, wherein the extracellular or extravesicullar binder structure comprises an affinity tag configured for interacting with the membrane recognition elements.
Clause 11: The artificial transmembrane protein according to any of the previous clauses wherein the transmembrane protein is label-free, in particular fluorescent label-free.
Clause 12: A cell, vesicle or cellular or vesicular component comprising an artificial transmembrane protein according to any of clauses 1 to 11 or a nucleic acid sequence encoding an artificial transmembrane protein according to any of clauses 1 to 10.
Clause 13: A recombinant nucleic acid molecule comprising at least one nucleic acid sequence encoding an artificial transmembrane protein according to any of clauses 1 to 10.
Clause 14: A vector, preferably a plasmid vector, comprising the recombinant nucleic acid molecule according to clause 12.
Clause 15: Use of a vector according to clause 13 for expressing an artificial transmembrane protein in vitro, comprising:
providing a cell and introducing a vector according to clause 13 in the cell, and expressing the artificial transmembrane protein.
Clause 16: The use of clause 14, further comprising the step of cleaving a signal peptide from the artificial transmembrane protein.
Clause 17: Biomolecular detection device for analyzing a cell, vesicle or a cellular or vesicular component comprising an artificial transmembrane protein according to any of clauses 1 to 10, the biomolecular detection device comprising an evanescent illuminator with an optical coupling unit configured for generating an evanescent field from coherent light with a predefined wavelength on a first surface of the evanescent illuminator, the first surface of the evanescent illuminator comprising a template nanopattern, containing a coherent arrangement of a plurality of predetermined lines along which membrane recognition elements for a binder structure of the artificial transmembrane protein, of the cell, vesicle or the cellular or vesicular component are arranged, wherein the membrane recognition elements are configured to bind the binder structure of the artificial transmembrane protein for forming a transmembrane nanopattern within the cell, vesicle or the cellular or vesicular component based on the template nanopattern of the evanescent illuminator, such that light of the evanescent field is scattered by the cell, vesicle or the cellular or vesicular component bound to the membrane recognition elements, and wherein the predetermined lines are arranged such that light scattered by the cell, vesicle or cellular or vesicular components bound to the membrane recognition elements constructively interferes at a predefined detection site with a difference in optical path length that is an integer multiple of the predefined wavelength of the coherent light.
Clause 18: Biomolecular detection device according to clause 17, wherein at least one cell, vesicle or cellular or vesicular component is bound via the binder structure of the artificial transmembrane protein to the membrane recognition elements.
Clause 19: Kit of parts comprising:
a. an artificial transmembrane protein according to any of clauses 1 to 11, or
   a cell according to clause 12, or
   a recombinant nucleic acid molecule according to clause 13, or
   a vector according to clause 14, and
b. a biomolecular detection device according to clause 16 or 17
Clause 20: The kit according to clause 19, further comprising a protein of interest configured for intracellular or intravesicular biomolecular interaction, wherein the protein of interest comprises a high-mass moiety, in particular a gold nanoparticle.
Clause 21: Label-free method for detecting intracellular or intravesicular biomolecular interactions in a cell, cellular component or a vesicle or vesicular component using a biomolecular detection device according to clause 16 or 17 the method comprising the steps:
   Providing a cell, cellular component or vesicle or vesicular component comprising an artificial transmembrane protein according to any of clauses 1 to 11;
   Applying the cell, cellular component or vesicle or vesicular component to the membrane recognition elements of the biomolecular detection device;
   Generating a beam of coherent light at a predefined beam generation location relative to the plurality of predetermined lines, the beam of coherent light having a predefined wavelength and being incident on the membrane recognition elements with the bound transmembrane protein in a manner that diffracted portions of the incident beam of coherent light constructively interfere at the predefined detection site relative to the plurality of predetermined lines with a difference in optical path length that is an integer multiple of the predefined wavelength of the coherent light to provide a signal representative of the membrane recognition elements with the artificial transmembrane protein of a cell, vesicle or cellular or vesicular component bound thereto at the predefined detection site;
   Measuring the signal representative for the membrane recognition elements with the artificial transmembrane protein of a cell, vesicle or cellular or vesicular component bound thereto.
Clause 22: The method according to clause 21, further comprising the step of providing a substance that interacts with the receptor structure of the artificial transmembrane protein or with intracellular or intravesicular component of the biomolecular interaction to be detected.
Clause 23: The method according to clause 21 or 22, wherein the cell or cellular comprising the artificial transmembrane protein is provided by in vitro transfecting a cell with a vector according to clause 14, expressing the artificial transmembrane protein in the cell and optionally removing parts of the membrane of the cell for providing the cellular component.

### BRIEF DESCRIPTION OF THE FIGURES

FIG. 1 shows a schematic representation of a biomolecular detection device according to an embodiment of the invention.

FIG. 2 shows a schematic cross-sectional view of a biomolecular detection device according to another embodiment of the invention.

FIG. 3a shows an artificial transmembrane protein according to an embodiment of the invention for detecting the intracellular interaction of the cAMP pathway.

FIG. 3b shows the signal expected from wild type cell during detection of the cAMP pathway as well as a control experiment with a G-protein knockout cell.

FIG. 3c shows the signal obtained from wild type cell during detection of the cAMP pathway as well as a control experiment with a G-protein knockout cell.

FIG. 4a shows two different artificial transmembrane proteins according to other embodiments of the invention for detecting the intracellular interaction of the ERK pathway.

FIG. 4b shows the signals associated with the artificial transmembrane proteins which is obtained during detection of the ERK pathway upon activation of the ERK pathway by a growth factor.

FIG. 4c shows the signals associated with the artificial transmembrane proteins which is obtained during detection of the ERK pathway upon inhibition of the ERK pathway downstream kinases.

FIGS. 5a and 5b show a schematic representation of a biomolecular detection device according to another embodiment of the invention.

FIGS. 6a and 6b show a schematic representation of a biomolecular detection device according to another embodiment of the invention.

### DETAILED DESCRIPTION

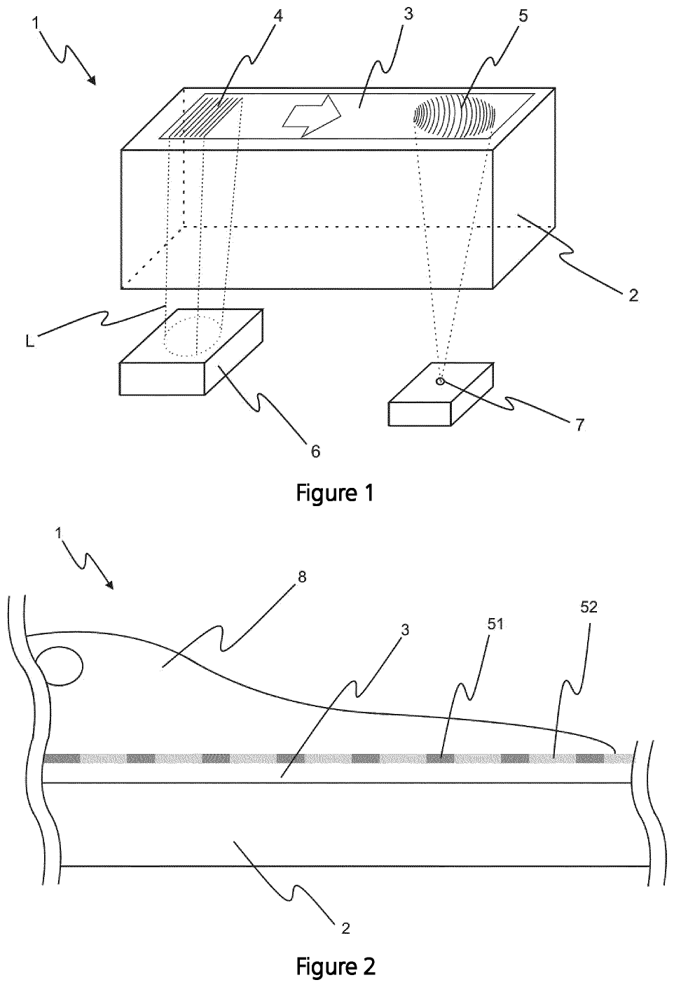

FIG. 1 shows a biomolecular detection device 1 according to an embodiment of the invention. The biomolecular detection device 1 comprises a carrier 2 with a surface on which planar waveguide 3 is arranged. The detection device further comprises optical coupler 4 for coupling coherent light L of a predefined wavelength into planar waveguide 3 such that coherent light propagates through the planar waveguide with an evanescent field of the coherent light propagating along a first surface of planar waveguide 3. The first surface of the planar waveguide is the surface facing away from carrier 2, i.e. the surface which is visible in FIG. 1. Besides optical coupler 4, the first surface of the waveguide 3 comprises template nanopattern 5, which contains a plurality of predetermined lines along which membrane recognition elements are arranged (not shown in FIG. 1). In general, a light source 6 is employed for providing an incoming beam of coherent light L towards optical coupler 4. Optical coupler 4 couples the coherent light L into planar waveguide 3 upon which coherent light L propagates in the direction of the arrow shown in FIG. 1 towards template nanopattern 5. If the membrane recognition elements are bound to a cell, vesicle or cellular or vesicular component via an artificial transmembrane protein according to any of the embodiments as described herein, light is scattered and due to the predefined lines, which are arranged such that light scattered by the cell, vesicle or cellular or vesicular components bound to the membrane recognition elements constructively interferes at a predefined detection site 7. In the particular embodiment shown, the predetermined lines are curved lines having a curvature configured such that light of the evanescent field scattered by the cell, vesicle or the cellular or vesicular component bound to the membrane recognition elements interferes at the predefined detection site 7. The distance between each of the membrane recognition elements of template nanopattern 5 and detection site 7 is referred to as the optical path length.

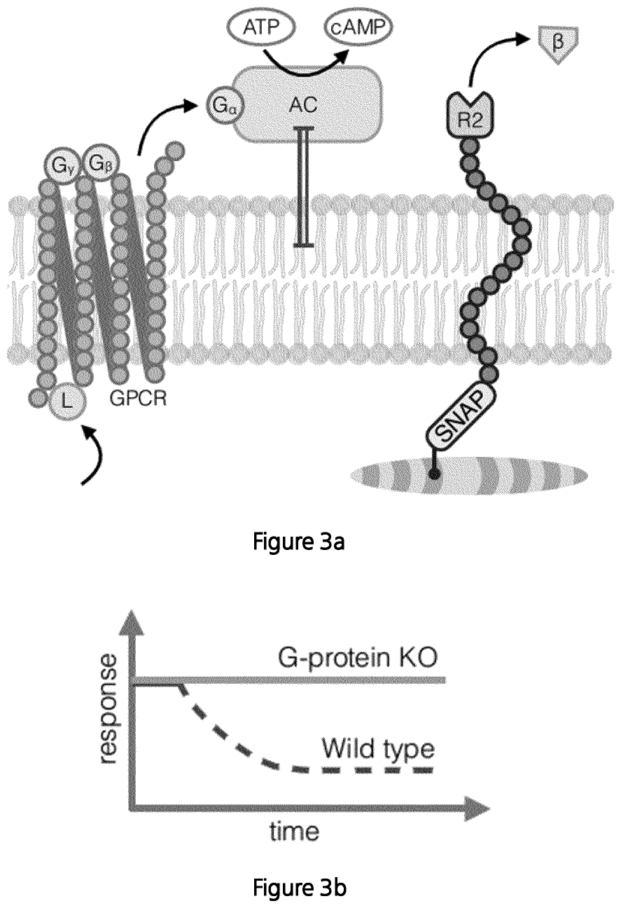

FIG. 2 illustrates a schematic cross-section of a biomolecular detection device 1 through template nanopattern 5 along the propagation direction of the light L through planar waveguide 3. Device 1 contains carrier 2 with waveguide 3 arranged on its surface. Arranged on top of the first surface of planar waveguide 3 is living cell 8. Furthermore, template nanopattern 5 comprises ridges 51 and grooves 52. Ridges 51 are areas along which membrane recognition elements are arranged. Grooves 52 are areas which do not contain any membrane recognition elements. Thus, a binder structure of a transmembrane protein of cell 8 can only bind to nanopattern 5 at a corresponding ridge. In general, the artificial transmembrane proteins as described herein are laterally diffusible within the cellular or vesicular membrane. Thus, the artificial transmembrane proteins laterally diffuse through the membrane, until they are in close proximity to a membrane recognition element, upon which a covalent bond may be formed for establishing the nanopattern within the cell, vesicle or cellular or vesicular component. It should be noted that the widths of the cell, nanopattern, waveguide and carrier do not provide any indication of their actual widths or the width ratios.

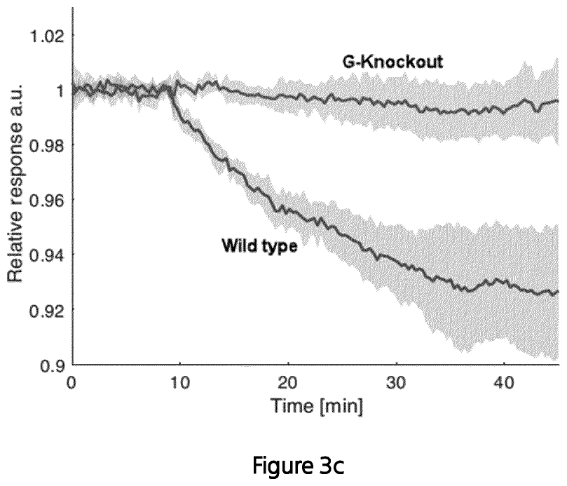

FIG. 3a shows a schematic representation of the cyclic AMP (cAMP) pathway, which is monitored with an artificial transmembrane protein according to an embodiment of the invention. The cAMP pathway is a G protein-coupled receptor-triggered signaling cascade which plays fundamental roles in cellular responses. In resting cells, G proteins are bound to the intracellular site of GPCRs. Upon activation of the GPCRs by external ligands, the a subunit of the G protein leaves the GPCR complex and activates the adenylate cyclase (AC), a membrane-bound enzyme with an intracellular active site. In turn, AC converts adenosine triphosphate (ATP) into cyclic adenosine monophosphate (cAMP), one of the most common second messengers of the cell. Moreover, AC plays a role in amplifying the signal, since the attack of an external ligand entails the synthesis of thousands second messenger molecules in a few seconds. The increased concentration of cAMP stimulates a cAMP-dependent protein kinase (PKA). PKA is a tetramer composed of two regulatory subunits (type I and type II), to which two cAMP molecules are bound for each subunit and by two (α and β) catalytic subunits. Binding of the cAMP molecules results in phosphorylation of the two catalytic subunits and, consequently, their dissociation from the two regulatory dimers. These phosphorylated subunits activate a wide range of targets that may be other signaling proteins as well as effector proteins. The final step of the cAMP pathway was chosen as a proof of concept for the working principle of the artificial transmembrane proteins according to the invention at hand. In order to probe the dissociation of the catalytic β subunit upon GPCR triggering, an artificial transmembrane with an intracellular or intravesicular domain with an intracellular or intravesicular receptor structure presenting a PKA type II-beta regulatory subunit on its cytosolic site was constructed (R2). As can be seen in FIG. 3b, a decrease of the signal intensity is to be expected, which is associated with the mass decrease of the complex as the catalytic β subunit dissociates (dashed line). At the same time, no change in signal should be detected in G protein knockout cells, as the absence of G protein results in no activation of the AC activity and therefore no dissociation of the catalytic β subunit. FIG. 3c shows the experimentally obtained signal for both HER293 cells (wild-type) and G protein knockout cells. While the signal of the knockout cells does not provide any change of signal, the wild type shows a decrease of the signal intensity upon dissociation of the catalytic β subunit.

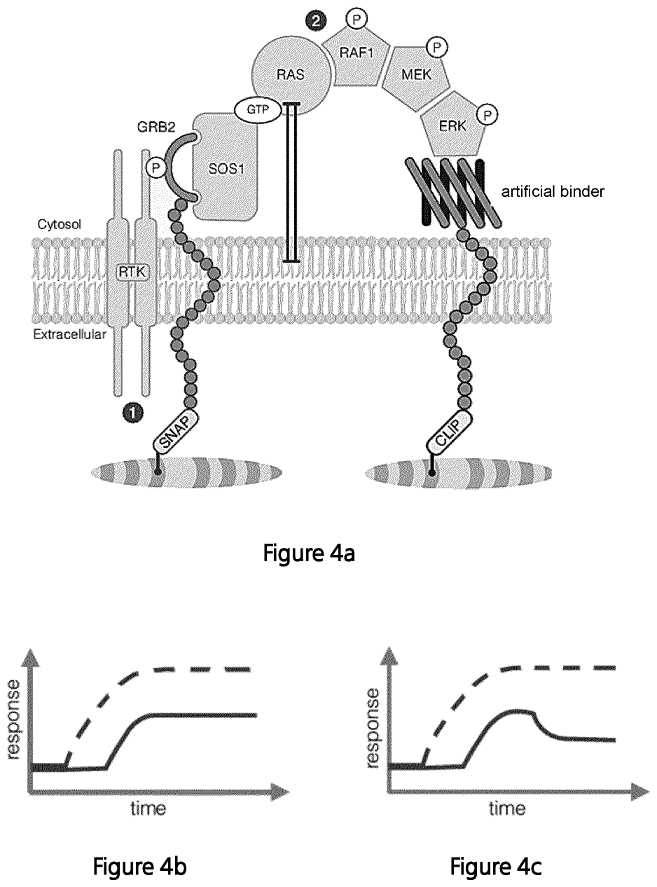

FIG. 4a shows a schematic representation of the ERK signaling pathway, which has been monitored by two different artificial transmembrane proteins. The ERK pathway is initiated by external ligands binding tyrosine kinase receptors (RTK), which are one of the most common types of enzyme-linked surface receptors. In general, an inactive RTK receptor is made up of two single-pass monomers that are activated and dimerized once the ligand binds to the extracellular domain. Dimerization involves transautophosphorylation (one monomer phosphorylates the other and vice versa) on specific tyrosine residues. Phosphorylated tyrosines increase the kinase activity of RTK and act as binding sites for specific intracellular proteins. The binding event occurs because these proteins are able to recognize the phosphorylated tyrosine and the conformation of the RTK around it. The proteins associated with RTK are in turn often associated with an adapter protein, Grb2, via an SH2 domain. Grb2 also has two other SH3 domains for interaction with other proteins. One of the SH3 domains often interacts with Sos, a protein which promotes the exchange of Ras-bound GDP by GTP. When Sos activates Ras, a monomeric GTPase which has GDP tied when inactive, replaces GDP with GTP, and in turn Ras further activates Sos in a positive feedback circuit. Ras activates Raf, which in turn, with a cascade mechanism, activates through Mek the extracellular signal-regulated kinases, commonly known as the ERK module. The ERK protein is found in either an activated or inactivated state, which corresponds to its phosphorylated and unphosphorylated form respectively. Phosphorylated ERK translocates into the cell nucleus and acts on gene regulatory proteins. The ERK module is often turned off by the ERK itself which inactivates Raf through negative feedback. In order to monitor the early-cascade effects of growth factors along the ERK pathway, a first artificial transmembrane protein is provided with a Grb2 protein as the receptor structure. Furthermore, downstream effects were monitored by means of a second artificial transmembrane protein comprising an artificial binder as the receptor structure for the phosphorylated ERK component. FIG. 4b shows the signals to be expected for the first artificial transmembrane protein (dashed line) and the second artificial transmembrane protein (continuous line) upon activation of the ERK cascade by a growth factor stimulation. FIG. 4c shows the signals to be expected for the first artificial transmembrane protein (Grb2, dashed line) and the second artificial transmembrane protein (artificial binder, continuous line) upon inhibition of the downstream kinases, which should result in a decrease of only the signal obtained from the second artificial transmembrane protein.

At first, it was tested whether the SH3 binding domain of the Grb2 protein as the receptor structure of the first artificial transmembrane protein was functional. In the event, cells comprising an artificial transmembrane protein with a Grb2 as the receptor structure and cells comprising an artificial transmembrane protein with a eYFP (enhanced yellow fluorescent protein) were treated with a protein specifically targeting Grb2. As can be seen from FIG. 4d, the signal remains within the baseline for about 20 minutes after the injection of the peptide. Thereafter, an increase in the response from the cells with the artificial transmembrane protein containing Grb2 as the receptor structure is observed, while the signal for the cells with the artificial transmembrane protein containing eYFP as the receptor structure remains constant at the baseline. The lag between injection and change in response is presumably due to the time required for the peptide to diffuse across the cell membrane and bind the Grb2 SH3 domain. Thus, the method allows for real time monitoring of an intracellular biomolecular interaction inside living cells. Furthermore, these results show that Grb2-targeting peptide specifically binds the Grb2 receptor structure inside living cells, while it does not bind a non-specific receptor structure such as the eYFP.

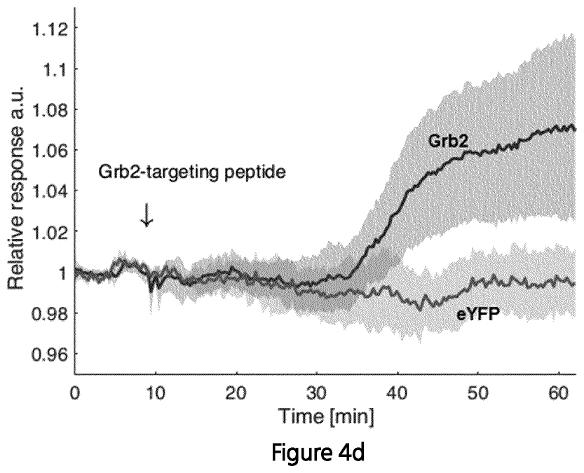

FIGS. 5a and 5b show a biomolecular detection device 1′ according to an embodiment of the invention. Device 1′ comprises an evanescent illuminator 2′ with an optical coupling unit 4′ configured for generating an evanescent field 9′ from coherent light with a predefined wavelength of a light source 6′ on a first surface of the evanescent illuminator 2′. The first surface of the evanescent illuminator 2′ comprises template nanopattern 5′ containing a coherent arrangement of a plurality of predetermined lines along which membrane recognition elements for a binder structure of a transmembrane protein 81 of cell 8′. As can be seen, by establishing a chemical bond between the membrane recognition element and the laterally diffusible transmembrane proteins 81, the nanopattern 5′ of the evanescent illuminator 2′ is transposed into the cell as a transmembrane nanopattern. In the embodiment in FIG. 5a, light source 6′ and detection unit 7′ are physically separate components, while in the embodiment shown in FIG. 5b, they are integral part of evanescent illuminator 2′.

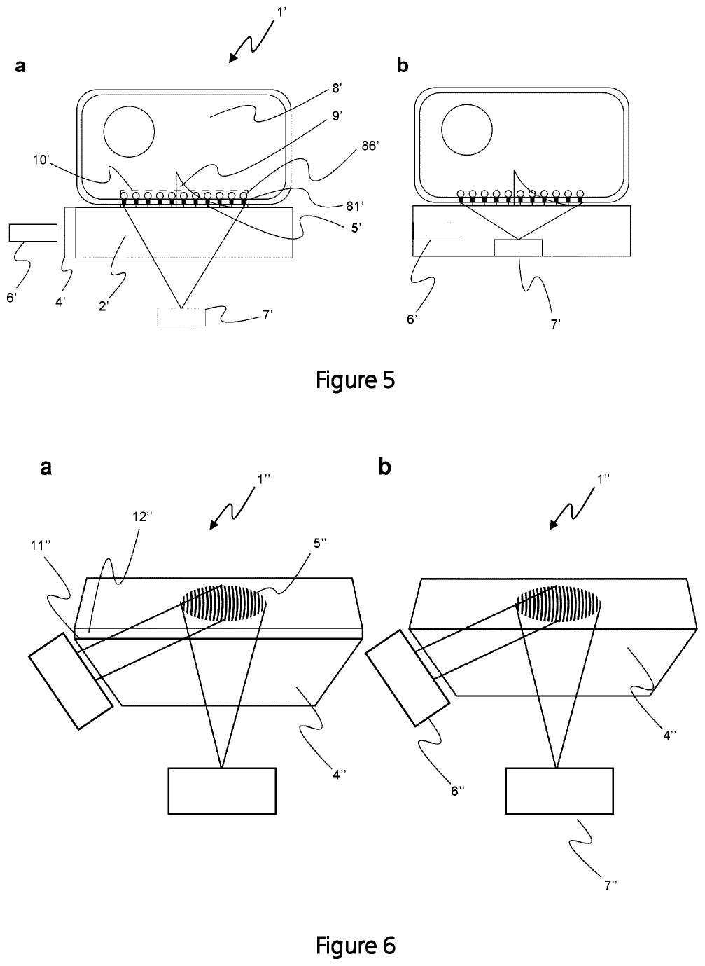

FIGS. 6a and 6b show an alternative embodiment of a biomolecular detection device 1″ according to the invention. The biomolecular detection device 1 comprises an evanescent illuminator which in these particular embodiments is a total internal reflection system configured for providing a beam of coherent light at the predetermined wavelength from light source 6″ and at a predetermined angle onto the first surface of the evanescent illuminator by means of the optical coupling unit 4″. The optical coupling unit in these embodiments is a prism. In the embodiment shown in FIG. 6a, the evanescent illuminator comprises an index matching medium 11″ such as index matching oils, DMSO, glycerol, water mixtures, hydrogels, etc., and a carrier slide 12″ which contains the template nanopattern 5″. Alternatively, as shown in FIG. 6b, the evanescent illuminator can be devoid of index matching medium 11″ and a carrier slide 12″. In this case, the nanopattern 5″ is directly provided on a first surface of the optical coupling unit 4″, i.e. the prism.

### Example

### Methods and Materials

DNA plasmids encoding for different artificial transmembrane proteins were purchased from Invitrogen GeneArt Gene Synthesis service by Thermo Fisher Scientific. All synthetic genes were assembled from synthetic oligonucleotides and/or PCR products and inserted into a pcDNA3.1(+) vector backbone. The plasmid DNA was purified from transformed bacteria, the concentration was determined by UV spectroscopy and the final constructs were verified by sequencing by the manufacturer. The sequence identity within the insertion sites was 100%. Plasmids were delivered in TE buffer at a concentration of ˜1 mg/ml and they were stored in working aliquots at −80° C.

The three signal peptide tested are specified in table 3.1, while additional amino acid sequences can be found in Table 1.

| Export sequence | Abbreviation | Amino acid code |
|---|---|---|
| Immunoglobin K | IgK | METDTLLLWVLLLWVPG |
| | | STGD |
| | | (SEQ ID NO: 1) |
| Optimized | IL2 | MRMQLLLLIALSLALVINS |
| Interleukin-2 | | (SEQ ID NO: 2) |
| Gaussia | GLuc | MGVKVLFALICIAVAEA |
| Luciferase | | (SEQ ID NO: 3) |

Table 2 shows the structural features of the plasmid vectors encoding for some of the artificial transmembrane proteins tested:

| Name | Plasmid structure |
|---|---|
| GLuc-eYFP | GLuc-SNAPf-PdgfrTM-Linker-eYFP |
| RV-GLuc-eYFP | GLuc-KKKK-eYFP-RvPdgfrTM-SNAPf |
| IL2-eYFP | IL2-SNAPf-PdgfrTM-Linker-eYFP |
| IgK-eYFP | IgK-SNAPf-PdgfrTM-Linker-eYFP |
| Grb2 | GLuc-SNAPf-PdgfrTM-Linker-Grb2 |
| R2ß | GLuc-SNAPf-PdgfrTM-Linker-R2β |

### Cell Culture and Transfection

HEK293 wild type and G-protein knockout cells were cultured in complete medium (DMEM medium containing 10% fetal bovine serum) at 37° C. in a cell incubator with 5% CO₂. For the generation of artificial transmembrane protein expressing cells, cells were transfected using Lipofectamine 3000 Transfection Reagent according to the manufacturer's protocol.

In order to establish stable cell lines, transiently transfected cells were grown in complete medium supplement by 1 mg/ml G418 for approximately 20 days. Afterwards, neomycin-resistant cells were stained using a SNAP-Surface 649 dye and selected by flow cytometry.

For fluorescence imaging, cells were seeded on a 24-glass bottom well plate at 50% confluence and transfected after 24 h as described previously. Transfection medium was replaced after 12 h with complete medium. Cells were imaged 12, 24, 36 and 48 hours after transfection using an Olympus FluoView FV3000 confocal laser scanning microscope. Prior to imaging, cells were incubated with SNAP-Surface 649 dye for 30 mins and then washed three times with warm PBS. During imaging, cells were kept at 37° C. with 5% CO2. The eYFP and SNAP-Surface 649 channels were acquired simultaneously with a 20× objective using 514 nm excitation/527 nm emission wavelengths for the green channel and 651 nm excitation/667 nm emission wavelengths for the red channel.

### Biomolecular Detection Device

Thin-film optical waveguides from Zeptosens were treated with a standard procedure (Gatterdam et al. Nature Nanotechnology, 12(11):1089-1095, September 2017) to coat them with a graft PAA-g-PEG polymer. The amine groups of the polymer are protected by photosensitive PhSNPPOC groups to allow for further processing. Afterwards, a reactive immersion lithography process described previously was used to pattern molograms on the optical waveguides. In brief, the polymer coated waveguide chip was mounted on a custom-made holder which allows for the alignment of a phasemask. After the phasemask was placed onto the holder, the chip was illuminated at 405 nm wavelength with a 2000 mJ/cm² dose in order to cleave off the photosensitive groups from the ridges of the nanopattern. The activated amine sites were incubated with either a BG-GLA-NHS or a BC-GLA-NHS substrate, for binding SNAP-tag or CLIP-tag respectively. Afterwards, full field illumination under UV light was performed in order to remove the remaining photosensitive groups from the grooves and the surroundings. The resulting amine groups were functionalized with a GRGDSPGSC (SEQ ID NO: 4) peptide

### Measurements

Cells were seeded to 100% confluency on the planar waveguide and let attach in complete medium for 2-3 hours while keeping the planar waveguide inside a cell incubator. Afterwards, medium was replaced with HEPES-buffered complete medium or HEPES-buffered HBSS adjusted to pH 7.4. Measurements were carried out on a F3000 ZeptoReader, kept at 35° C. with 5% CO2. Images were acquired every 15 seconds using the 635 nm laser with an exposure time comprised between 0.1 s and 1 s. Pharmacological manipulation was done on chip after a 10 minutes baseline (30 images) was established.

Amino Acid Sequences

| Name | Abbreviation | Sequence |
|---|---|---|
| TM domain | PdgfrTM | AVGQDTQEVIVVPHSLPFKVVVISAILALVVLTIISLIILIMLWQKKPR |
| | | (SEQ ID NO: 5) |
| Reversed TM | RvPdgfrTM | RPKKQWLMILIILSIITLVVLALIASIVVVKPFPLSHPVVIVEQTDQGV |
| domain | | A (SEQ ID NO: 6) |
| Flexible linker | Linker | GGGGSGGGGSGSAGSAAGSGEFGGGGSGGGGS (SEQ ID NO: 7) |
| SNAP-tag | SNAPf | MDKDCEMKRTTLDSPLGKLELSGCEQGLHRIIFLGKGTSAADAVEVPAP |
| | | AAVLGGPEPLMQATAWLNAYFHQPEAIEEFPVPALHHPVFQQESFTRQV |
| | | LWKLLKVVKFGEVISYSHLAALAGNPAATAAVKTALSGNPVPILIPCHR |
| | | VVQ GDLDVGGYEGGLAVKEWLLAHEGHRLGKPGLG |
| | | (SEQ ID NO: 8) |
| CLIP-tag | CLIP | MDKDCEMKRTTLDSPLGKLELSGCEQGLHRIIFLGKGTSAADAVEVPAP |
| | | AAVLGGPEPLIQATAWLNAYFHQPEAIEEFPVPALHHPVFQQESFTRQV |
| | | LWKLLKVVKFGEVISESHLAALVGNPAATAAVNTALDGNPVPILIPCHR |
| | | VVQ GDSDVGPYLGGLAVKEWLLAHEGHRLGKPGLG |
| | | (SEQ ID NO: 9) |
| eYFP | eYFP | MVSKGEELFTGVVPILVELDGDVNGHKFSVSGEGEGDATYGKLTLKFIC |
| | | TTGKLPVPWPTLVTTFGYGLQCFARYPDHMKQHDFFKSAMPEGYVQERT |
| | | IFFKDDGNYKTRAEVKFEGDTLVNRIELKGIDFKEDGNILGHKLEYNYN |
| | | SHNVYIMADKQKNGIKVNFKIRHNIEDGSVQLADHYQQNTPIGDGPVLL |
| | | PDNHYLSYQSALSKDPNEKRDHMVLLEFVTAAGITLGMDELYK |
| | | (SEQ ID NO: 10) |
| Grb2 | Grb2 | MEAIAKYDFKATADDELSFKRGDILKVLNEECDQNWYKAELNGKDGFIP |
| | | KNYIEMKPHPWFFGKIPRAKAEEMLSKQRHDGAFLIRESESAPGDFSLS |
| | | VKFGNDVQHFKVLRDGAGKYFLWVVKFNSLNELVDYHRSTSVSRNQQIF |
| | | LRDIEQVPQQPTYVQALFDFDPQEDGELGFRRGDFIHVMDNSDPNWWKG |
| | | ACHGQTGMFPRNYVTPVNRNVFGNDVQHFKVLRDGAGKYFLWVVKFNSL |
| | | NELVDYHRSTSVSRNQQIFLRDIEQVPQQPTYVQALFDFDPQEDGELGF |
| | | RRGDFIHVMDNSDPNWWKGACHGQTGMFPRNYVTPVNRNVFGNDVQHFK |
| | | VLRDGAGKYFLWVVKFNSLNELVDYHRSTSVSRNQQIFLRDIEQVPQQP |
| | | TYVQALFDFDPQEDGELGFRRGDFIHVMDNSDPNWWKGACHG QTGMFP |
| | | RNYVTPVNRNV (SEQ ID NO: 11) |
| PKA type II | R2ß | MSIEIPAGLTELLQGFTVEVLRHQPADLLEFALQHFTRLQQENERKGTA |
| regulatory | | RFGHEGRTWGDLGAAAGGGTPSKGVNFAEEPMQSDSEDGEEEEAAPADA |
| subunit | | GAFNAPVINRFTRRASVCAEAYNPDEEEDDAESRIIHPKTDDQRNRLQE |
| | | ACKDILLFKNLDPEQMSQVLDAMFEKLVKDGEHVIDQGDDGDNFYVIDR |
| | | GTFDIYVKCDGVGRCVGNYDNRGSFGELALMYNTPRAATITATSPGALW |
| | | GLDRVTFRRIIVKNNAKKRKMYESFIESLPFLKSLEFSERLKVVDVIGT |
| | | KVYNDGEQIIAQGDSADSFFIVESGEVKITMKRKGKSEVEENGAVEIAR |
| | | CSRGQYFGELALVTNKPRAASAHAIGTVKCLAMDVQAFERLLGPCMEIM |
| | | KRNIATYEEQLVALFGTNMDIVEPTA (SEQ ID NO: 12) |
| Grb2-targeting | P6 | KKWKMRRNPFWIKIQRC- |
| peptide | | CGIRVVDNSPPPPLPPRRRRSAPSPTRV-amide |
| | | (SEQ ID NO: 13) |

1. An artificial transmembrane protein for use in a biomolecular detection device for detecting intracellular or intravesicular biomolecular interactions, the artificial transmembrane protein comprising an extracellular or extravesicular binder structure, a hydrophobic transmembrane domain and an intracellular or intravesicular domain with an intracellular or intravesicular receptor structure, wherein the receptor structure is configured to interact with an intracellular or intravesicular component of the biomolecular interaction to be detected and wherein the extracellular or extravesicular binder structure is configured to bind to membrane recognition elements arranged along a plurality of predetermined lines of the biomolecular detection device.

2. The artificial transmembrane protein according to claim 1, further comprising a linker domain configured to facilitate the interaction between the intracellular or intravesicular receptor structure and the intracellular or intravesicular component of the biomolecular interaction to be detected, wherein the linker domain is arranged between the intracellular or intravesicular receptor structure and the hydrophobic transmembrane domain.

3. The artificial transmembrane protein according to claim 1, wherein the extracellular or extravesicular binder structure is configured to establish a covalent bond to the membrane recognition elements arranged along a plurality of predetermined lines of the biomolecular detection device.

4. The artificial transmembrane protein according to claim 1, wherein the extracellular or extravesicular binder structure comprises a nucleophile.

5. The artificial transmembrane protein according to claim 1, wherein the extracellular or extravesicular binder structure is a SNAP tag or a CLIP tag.

6. The artificial transmembrane protein according to claim 1, wherein the artificial transmembrane protein is
(a) of type I, wherein the intracellular or intravesicular domain is arranged adjacent to the C terminus and the extracellular or extravesicular domain is arranged adjacent to the N terminus; or
(b) of type II wherein the intracellular or intravesicular domain is arranged adjacent to the N terminus and the extracellular or extravesicular domain is arranged adjacent to the C terminus.

7. The artificial transmembrane protein according to claim 6, wherein the intracellular or intravesicular domain comprises a higher amount of positively charged amino acid residues than the extracellular or extravesicular domain.

8. The artificial transmembrane protein according to claim 1, wherein the intracellular or intravesicular receptor structure is a designed receptor or other functional molecule.

9. The artificial transmembrane protein according to claim 1, further comprising a cleavable signal peptide adjacent the N terminus of the artificial transmembrane protein for interaction with a protein transport system and for controlling translocation of the artificial transmembrane protein.

10. The artificial transmembrane protein according to claim 1, wherein the extracellular or extravesicullar binder structure comprises an affinity tag configured for interacting with the membrane recognition elements.

11. The artificial transmembrane protein according to claim 1, wherein the transmembrane protein is label-free, in particular fluorescent label-free.

12. A cell, vesicle or cellular or vesicular component comprising an artificial transmembrane protein, or a nucleic acid sequence encoding an artificial transmembrane protein, according to claim 1.

13. A recombinant nucleic acid molecule comprising at least one nucleic acid sequence encoding an artificial transmembrane protein according to any of claim 1.

14. A vector, comprising the recombinant nucleic acid molecule according to claim 13.

15. A method of expressing an artificial transmembrane protein in vitro, comprising:
providing a cell and introducing a vector according to claim 14 in the cell, and expressing the artificial transmembrane protein.

16. A bimolecular detection device for analyzing a cell, vesicle, or a cellular or vesicular component comprising an artificial transmembrane protein according to claim 1, the biomolecular detection device comprising an evanescent illuminator with an optical coupling unit configured for generating an evanescent field from coherent light (L) with a predefined wavelength on a first surface of the evanescent illuminator, the first surface of the evanescent illuminator comprising a template nanopattern, containing a coherent arrangement of a plurality of predetermined lines along which membrane recognition elements for a binder structure of the artificial transmembrane protein, of the cell, vesicle or the cellular or vesicular component are arranged, wherein the membrane recognition elements are configured to bind the binder structure of the artificial transmembrane protein for forming a transmembrane nanopattern within the cell, vesicle or the cellular or vesicular component based on the template nanopattern of the evanescent illuminator, such that light of the evanescent field is scattered by the cell, vesicle or the cellular or vesicular component bound to the membrane recognition elements, and wherein the predetermined lines are arranged such that light scattered by the cell, vesicle or cellular or vesicular components bound to the membrane recognition elements constructively interferes at a predefined detection site with a difference in optical path length that is an integer multiple of the predefined wavelength of the coherent light (L).

17. A kit comprising:
a. an artificial transmembrane protein comprising an extracellular or extravesicular binder structure, a hydrophobic transmembrane domain and an intracellular or intravesicular domain with an intracellular or intravesicular receptor structure, wherein the receptor structure is configured to interact with an intracellular or intravesicular component of the biomolecular interaction to be detected and wherein the extracellular or extravesicular binder structure is configured to bind to membrane recognition elements arranged along a plurality of predetermined lines of a biomolecular detection device, or
a cell comprising an artificial transmembrane protein, or a nucleic acid sequence encoding an artificial transmembrane protein, the transmembrane protein comprising an extracellular or extra vesicular binder structure, a hydrophobic transmembrane domain and an intracellular or intravesicular domain with an intracellular or intravesicular receptor structure, wherein the receptor structure is configured to interact with an intracellular or intravesicular component of the biomolecular interaction to be detected and wherein the extracellular or extravesicular hinder structure is configured to bind to membrane recognition elements arranged along a plurality of predetermined lines of a biomolecular detection device, or
a recombinant nucleic acid molecule comprising at least one nucleic acid sequence encoding an artificial transmembrane protein an extracellular or extravesicular hinder structure, a hydrophobic transmembrane domain and an intracellular or intravesicular domain with an intracellular or intravesicular receptor structure, wherein the receptor structure is configured to interact with an intracellular or intravesicular component of the biomolecular interaction to be detected and wherein the extracellular or extravesicular hinder structure is configured to bind to membrane recognition elements arranged along a plurality of predetermined lines of a biomolecular detection device, or
a vector comprising a recombinant nucleic acid molecule comprising at least one nucleic acid sequence encoding an artificial transmembrane protein an extracellular or extravesicular binder structure, a hydrophobic transmembrane domain and an intracellular or intravesicular domain with an intracellular or intravesicular receptor structure, wherein the receptor structure is configured to interact with an intracellular or intravesicular component of the biomolecular interaction to be detected and wherein the extracellular or extravesicular binder structure is configured to bind to membrane recognition elements arranged along a plurality of predetermined lines of a biomolecular detection device; and
b. a biomolecular detection device according to claim 16; and optionally
c. a protein of interest configured for intracellular or intravesicular biomolecular interaction, wherein the protein of interest comprises a high-mass moiety.

18. A label-free method for detecting intracellular or intravesicular biomolecular interactions in a cell, cellular component, or a vesicle or vesicular component comprising:
providing a cell, cellular component, or vesicle or vesicular component comprising an artificial transmembrane protein according to claim 1;
applying the cell, cellular component, or vesicle or vesicular component to membrane recognition elements of a biomolecular detection device comprising:
an evanescent illuminator with an optical coupling unit configured for generating an evanescent field from coherent light (L) with a predefined wavelength on a first surface of the evanescent illuminator, the first surface of the evanescent illuminator comprising a template nanopattern, containing a coherent arrangement of a plurality of predetermined lines along which membrane recognition elements for a binder structure of the artificial transmembrane protein, of the cell, vesicle or the cellular or vesicular component are arranged, wherein the membrane recognition elements are configured to bind the hinder structure of the artificial transmembrane protein for forming a transmembrane nanopattern within the cell, vesicle or the cellular or vesicular component based on the template nanopattern of the evanescent illuminator, such that light of the evanescent field is scattered by the cell, vesicle or the cellular or vesicular component bound to the membrane recognition elements, and wherein the predetermined lines are arranged such that light scattered by the cell, vesicle or cellular or vesicular components bound to the membrane recognition elements constructively interferes at a predefined detection site with a difference in optical path length that is an integer multiple of the predefined wavelength of the coherent light (L);
generating a beam of coherent light at a predefined beam generation location relative to the plurality of predetermined lines, the beam of coherent light having a predefined wavelength and being incident on the membrane recognition elements with the bound transmembrane protein in a manner that diffracted portions of the incident beam of coherent light constructively interfere at the predefined detection site relative to the plurality of predetermined lines with a difference in optical path length that is an integer multiple of the predefined wavelength of the coherent light to provide a signal representative of the membrane recognition elements with the artificial transmembrane protein of a cell, vesicle or cellular or vesicular component bound thereto at the predefined detection site; and
measuring the signal representative for the membrane recognition elements with the artificial transmembrane protein of a cell, vesicle, or cellular or vesicular component bound thereto.


SEQUENCE LISTING (13 sequences)

SEQ ID NO: 1 - protein, 21 aa, Artificial Sequence
  Immunoglobin K
METDTLLLWVLLLWVPGSTGD

SEQ ID NO: 2 - protein, 19 aa, Artificial Sequence
  Optimized Interleukin-2
MRMQLLLLIALSLALVTNS

SEQ ID NO: 3 - protein, 17 aa, Artificial Sequence
  Gaussia Luciferase
MGVKVLFALICIAVAEA

SEQ ID NO: 4 - protein, 9 aa, Artificial Sequence
  Functional peptide
GRGDSPGSC

SEQ ID NO: 5 - protein, 49 aa, Artificial Sequence
  TM domain
AVGQDTQEVIVVPHSLPFKVVVISAILALVVLTIISLIILIMLWQKKPR

SEQ ID NO: 6 - protein, 49 aa, Artificial Sequence
  Reversed TM domain
RPKKQWLMILIILSIITLVVLALIASIVVVKFPLSHPVVIVEQTDQGVA

SEQ ID NO: 7 - protein, 32 aa, Artificial Sequence
  Flexible linker
GGGGSGGGGSGSAGSAAGSGEFGGGGSGGGGS

SEQ ID NO: 8 - protein, 182 aa, Artificial Sequence
  SNAP-tag
MDKDCEMKRTTLDSPLGKLELSGCEQGLHRIIFLGKGTSAADAVEVPAPAAVLGGPEPLMQATAWLNAYFHQPEAIEEFPVPALHHPVFQQESFTRQVLWKLLKVVKFGEVISYSHLAALAGNPAATAAVKTALSGNPVPILIPCHRVVQGDLDVGGYEGGLAVKEWLLAHEGHRLGKPGLG

SEQ ID NO: 9 - protein, 182 aa, Artificial Sequence
  CLIP-tag
MDKDCEMKRTTLDSPLGKLELSGCEQGLHRIIFLGKGTSAADAVEVPAPAAVLGGPEPLIQATAWLNAYFHQPEAIEEFPVPALHHPVFQQESFTRQVLWKLLKVVKFGEVISESHLAALVGNPAATAAVNTALDGNPVPILIPCHRVVQGDSDVGPYLGGLAVKEWLLAHEGHRLGKPGLG

SEQ ID NO: 10 - protein, 239 aa, Artificial Sequence
  eYFP
MVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 11 - protein, 451 aa, Artificial Sequence
  Grb2
MEAIAKYDFKATADDELSFKRGDILKVLNEECDQNWYKAELNGKDGFIPKNYIEMKPHPWFFGKIPRAKAEEMLSKQRHDGAFLIRESESAPGDFSLSVKFGNDVQHFKVLRDGAGKYFLWVVKFNSLNELVDYHRSTSVSRNQQIFLRDIEQVPQQPTYVQALFDFDPQEDGELGFRRGDFIHVMDNSDPNWWKGACHGQTGMFPRNYVTPVNRNVFGNDVQHFKVLRDGAGKYFLWVVKFNSLNELVDYHRSTSVSRNQQIFLRDIEQVPQQPTYVQALFDFDPQEDGELGFRRGDFIHVMDNSDPNWWKGACHGQTGMFPRNYVTPVNRNVFGNDVQHFKVLRDGAGKYFLWVVKFNSLNELVDYHRSTSVSRNQQIFLRDIEQVPQQPTYVQALFDFDPQEDGELGFRRGDFIHVMDNSDPNWWKGACHGQTGMFPRNYVTPVNRNV

SEQ ID NO: 12 - protein, 418 aa, Artificial Sequence
  PKA type II regulatory subunity
MSIEIPAGLTELLQGFTVEVLRHQPADLLEFALQHFTRLQQENERKGTARFGHEGRTWGDLGAAAGGGTPSKGVNFAEEPMQSDSEDGEEEEAAPADAGAFNAPVINRFTRRASVCAEAYNPDEEEDDAESRIIHPKTDDQRNRLQEACKDILLFKNLDPEQMSQVLDAMFEKLVKDGEHVIDQGDDGDNFYVIDRGTFDIYVKCDGVGRCVGNYDNRGSFGELALMYNTPRAATITATSPGALWGLDRVTFRRIIVKNNAKKRKMYESFIESLPFLKSLEFSERLKVVDVIGTKVYNDGEQIIAQGDSADSFFIVESGEVKITMKRKGKSEVEENGAVEIARCSRGQYFGELALVTNKPRAASAHAIGTVKCLAMDVQAFERLLGPCMEIMKRNIATYEEQLVALFGTNMDIVEPTA

SEQ ID NO: 13 - protein, 45 aa, Artificial Sequence
  Grb-2 targetign peptide
KKWKMRRNPFWIKIQRCCGIRVVDNSPPPPLPPRRRRSAPSPTRV